(19) United States
(12) Patent Application Publication
(10) Pub. No.: US 2004/0191905 A1
(43) Pub. Date: Sep. 30, 2004

(54) Modulation of HIV replication by RNA interference

(75) Inventors: Mario Stevenson, Shrewsbury, MA (US); Jean-Marc Jacque, Auburn, MA (US)

Correspondence Address: LAHIVE & COCKFIELD, LLP., 28 STATE STREET, BOSTON, MA, 02109, US

(73) Assignee: UNIVERSITY OF MASSACHUSETTS, Worcester, MA

(21) Appl. No.: 10/722,689

(22) Filed: Nov. 24, 2003

Related U.S. Application Data
(60) Provisional application No. 60/444,893, filed on Feb. 4, 2003.
(60) Provisional application No. 60/428,631, filed on Nov. 22, 2002.

(51) Int. Cl. A61K 48/00; C07H 21/02

(52) U.S. Cl. 435/375; 514/44; 536/23.1

(57) ABSTRACT

Disclosed herein are small interfering RNAs (siRNAs), and vectors encoding one or more siRNAs (including short hairpin siRNAs), that are sufficiently homologous to a portion of the HIV genome to mediate RNA interference in vivo. Also disclosed are methods wherein siRNAs, or vectors encoding siRNAs, are administered to prevent or inhibit HIV infection in a subject, cell or tissue. Knockout and/or knockdown cells or organisms are also disclosed that utilize the siRNAs or vectors of the present invention.

RELATED APPLICATIONS

This application is related to U.S. Provisional Patent Application Serial No. 60/428,631, filed Nov. 22, 2002, and U.S. Provisional Patent Application Serial No. 60/444,893, filed Feb. 4, 2003, both entitled “Modulation of HIV Replication by RNA Interference”, the entire contents of which are incorporated herein by this reference.

### GOVERNMENT SUPPORT

Work described herein was supported by Federal Grant Nos. RR 11589 and AI 37475 awarded by the National Institutes of Health. The Government has certain rights in this invention.

### BACKGROUND OF THE INVENTION

RNA interference (RNAi) is a ubiquitous mechanism of gene regulation in plants and animals in which target mRNAs are degraded in a sequence-specific manner (Sharp, P. A., Genes Dev. 15, 485-490 (2001); Hutvagner, G. & Zamore, P. D., Curr. Opin. Genet. Dev. 12, 225-232 (2002); Fire, A., et al., Nature 391, 806-811 (1998); Zamore, P., et al., Cell 101, 25-33 (2000)). The natural RNA degradation process is initiated by the dsRNA-specific endonuclease Dicer, which promotes processive cleavage of long dsRNA precursors into double-stranded fragments between 21 and 25 nucleotides long, termed small interfering RNA (siRNA) (Zamore, P., et al., Cell 101, 25-33 (2000); Elbashir, S. M., et al., Genes Dev. 15, 188-200 (2001); Hammond, S. M., et al., Nature 404, 293-296 (2000); Bernstein, E., et al., Nature 409, 363-366 (2001)). siRNAs are incorporated into a large protein complex that recognizes and cleaves target mRNAs (Nykanen, A., et al., Cell 107, 309-321 (2001). It has been reported that introduction of dsRNA into mammalian cells does not result in efficient Dicer-mediated generation of siRNA and therefore does not induce RNAi (Caplen, N. J., et al., Gene 252, 95-105 (2000); Ui-Tei, K., et al., FEBS Lett. 479, 79-82 (2000)). The requirement for Dicer in maturation of siRNAs in cells can be bypassed by introducing synthetic 21-nucleotide siRNA duplexes, which inhibit expression of transfected and endogenous genes in a variety of mammalian cells (Elbashir, et al., Nature 411: 494-498 (2001)).

Human immunodeficiency virus (HIV) has been implicated as the primary cause of the slowly degenerative disease of the immune system termed acquired immune deficiency syndrome (AIDS). AIDS was first reported in the United States in 1981 and has since become a major worldwide epidemic. According to the National Institute of Allergy and Infectious Diseases (NIAID), more than 790,000 cases of AIDS have been reported in the United States since 1981, and as many as 900,000 Americans may be infected with HIV. According to the December 2002 AIDS Epidemic Update released by the World Health Organization in collaboration with the United Nations, more than 5 million people worldwide will have contracted the AIDS virus in 2002, bringing the total number of those infected to 42 million (3.2 million are children under the age of 15). A total of 3.1 million people, 610,000 of them under the age of 15, will have died of HIV/AIDS related causes in 2002.

HIV infection leads to depletion of lymphocytes which inevitably leads to opportunistic infections, neoplastic growth and eventual death. Many antiviral drugs have been developed to inhibit HIV infection and replication including non-nucleoside reverse transcriptase inhibitors (e.g., delvaridine, nevirapine, and efravirenz), and protease inhibitors, (e.g., ritonavir, saquinivir, and indinavir), that are often prescribed in combination with other antiretroviral drugs. Over time, however, the HIV virus develops resistance to these therapeutic treatments, particularly after a prolonged drug regimen wherein there is relatively small drop in viral load, followed by a rise in amount of detectable virus in blood. Consequently, new treatments are desperately needed.

### SUMMARY OF THE INVENTION

The present invention provides a new therapeutic approach for preventing virus replication or infection in a subject. In a preferred embodiment, the virus is a retrovirus. The virus can be, e.g., HIV virus, Human T-cell Lukemia Virus (HTLV), and viral Hepatitis, including types and subtypes of these viruses, e.g., HIV-1, HIV-2, Hepatitis A, B, C, D or E, or HTLV-BLV. In a particularly preferred embodiment, the virus is HIV. The present invention is based, at least in part, on the discovery that one or more siRNAs targeted to various regions of the viral genome (e.g., HIV-1 genome) inhibit viral replication in human cell lines and primary lymphocytes. It has further been discovered that synthetic siRNA duplexes, and even more interestingly, plasmid-derived siRNAs, e.g., shRNAs, inhibit viral infection by specifically degrading genomic RNA, thereby preventing its establishment into the host cell and/or its replication in the host cell.

The invention further contemplates plasmids that express multiple siRNAs, which can be used to target multiple regions of the viral (e.g., HIV) genome to mediate RNAi. The use of multiple siRNAs mediates RNAi despite mutations in the genome that may cause one or more of the siRNAs to be insufficiently homologous to mediate RNAi.

Also discovered and demonstrated herein is the utility of RNAi for modulating the viral (e.g., HIV) replication cycle, and that genomic RNA, as it exists within a nucleoprotein reverse-transcription complex, is amenable to siRNA-mediated degradation. Accordingly, the methods of the present invention can be used to promote the degradation or inhibit the synthesis of genomic RNA before and/or after integration in the host cell genome. Furthermore, the present invention may be used to treat individuals as the virus mutates by synthesizing siRNAs that match the mutated viral genome.

Accordingly, the present invention provides new compositions for RNA interference and methods of use thereof. In particular, the invention provides siRNAs, and plasmid expressed-siRNAs for mediating RNAi in vitro and in vivo. Methods for using said siRNAs are also provided. In particular, therapeutic and prophylactic methods are featured.

Other features and advantages of the invention will be apparent from the following detailed description and claims.

### BRIEF DESCRIPTION OF THE DRAWINGS

FIGS. 1A-E illustrate that small interfering RNAs inhibit late events in HIV replication by promoting degradation of HIV-1 RNA. FIG. 1A is a schematic representation of HIV targets of siRNAs used in the examples. Small interfering RNAs completely homologous to the target HIV sequence (HIV_{NL-GFP}) are shown in ovals and those harboring nucleotide mismatches are shown in circles. FIG. 1B is a bar graph depicting the effect of siRNAs on HIV-1 particle production as determined by RT activity. FIG. 1C includes images of SDS-polyacrylamide gels depicting levels of total and active (phosphorylated) PKR levels in siRNA-transfected Magi cells. FIG. 1D includes a schematic representation, chart, and images of an agarose gel, that illustrate that small interfering RNAs mediate sequence-specific HIV RNA degradation. The presence of HIV_{NL-GFP} or HIV_{YU-2} RNA was determined by RT-PCR using HIV Nef-specific primers. Because of the GFP insertion in HIV_{NL-GFP} Nef, RNAs originating from HIV_{NL-GFP} are 710 nucleotides larger than those originating from HIV_{YU-2}. M is the molecular weight marker (100 bp ladder, New England Biolabs). FIG. 1E depicts a series of images of bright field illumination and fluorescence images that illustrate the effect of siRNAs on HIV expression in activated primary PBLs.

FIGS. 2A-F illustrate that small interfering RNAs block early events in HIV replication by promoting degradation of incoming genomic HIV RNA. FIG. 2A is a schematic representation of the experimental design used to investigate whether siRNAs were able to direct the specific degradation of HIV genomic RNA. FIG. 2B is a bar graph depicting the levels of trypsin-resistant HIV gag p24 in siRNA-transfected cells. The dash indicates no siRNA transfected into the cells. FIG. 2C is a schematic representation of the strategy for analysis of viral nucleic acid intermediates formed early after HIV infection. Major cDNA intermediates in viral reverse transcription are indicated. Horizontal lines indicate viral RNA, horizontal arrows indicate viral cDNA, and open circles and squares indicate primer-binding sites for initiation of minus-strand synthesis and polypurine tracts for plus-strand synthesis, respectively. HIV-specific primers (half-arrows) are shown next to the earliest cDNA intermediate they amplify. Integrated (proviral) HIV DNA was amplified using an HIV LTR-specific primer (Rc) and a primer directed to alu repeats (filled circles) within flanking cellular DNA. FIG. 2D is an image of an agarose gel illustrating the effect of siRNAs on genomic viral RNA. FIG. 2E is a series of bar graphs depicting the effect of siRNAs on formation of HIV-1 reverse transcription (RT) intermediates. FIG. 2F is an image of an agarose gel depicting reduced levels of viral integration in siRNA-transfected cells.

FIGS. 3A-D illustrate inhibition of HIV replication by siRNAs derived from plasmid DNA templates. FIG. 3A is a schematic representation of the strategy for production of hairpin siRNAs from plasmid vectors. Linearization of each construct with BstBI and transfection into cells with a plasmid expressing T7 RNA polymerase (Pol) predicts the expression of a hairpin RNA with a 19-bp self-complementary vif stem and non-base-paired loops of 3, 5 and 7 nucleotides. FIG. 3B is a bar graph depicting the effect of plasmid derived vif hairpin siRNAs on HIV particle production. T1 ΔVif is identical to plasmids that express vif hairpin except that it lacks self-complementary vif sequences. FIG. 3C is an image of an agarose gel illustrating that vif hairpin siRNAs promote degradation of HIV RNA. PCR products amplified from HIV_{NL-GFP} DNA served as a control. FIG. 3D is a series of images of bright field illumination and fluorescence images that illustrate inhibition of HIV-1 expression by vif hairpin siRNAs in primary PBLs.

### DETAILED DESCRIPTION OF THE INVENTION

So that the invention may be more readily understood, certain terms are first defined.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g, by DNA replication or transcription of DNA, respectively). RNA can be post-transcriptionally modified. DNA and RNA can also be chemically synthesized.

The term “RNA interference” (“RNAi”) refers to selective intracellular degradation of RNA (also referred to as gene silencing). RNAi occurs in cells naturally to remove foreign RNAs (e.g., viral RNAs). Natural RNAi proceeds via dicer-directed fragmentation of precursor dsRNA which direct the degradation mechanism to other cognate RNA sequences. Alternatively, RNAi can be initiated by the hand of man, for example, by transfection of small interfering RNAs (siRNAs) or production of siRNAs (e.g., from a plasmid or transgene), to silence the expression of target genes.

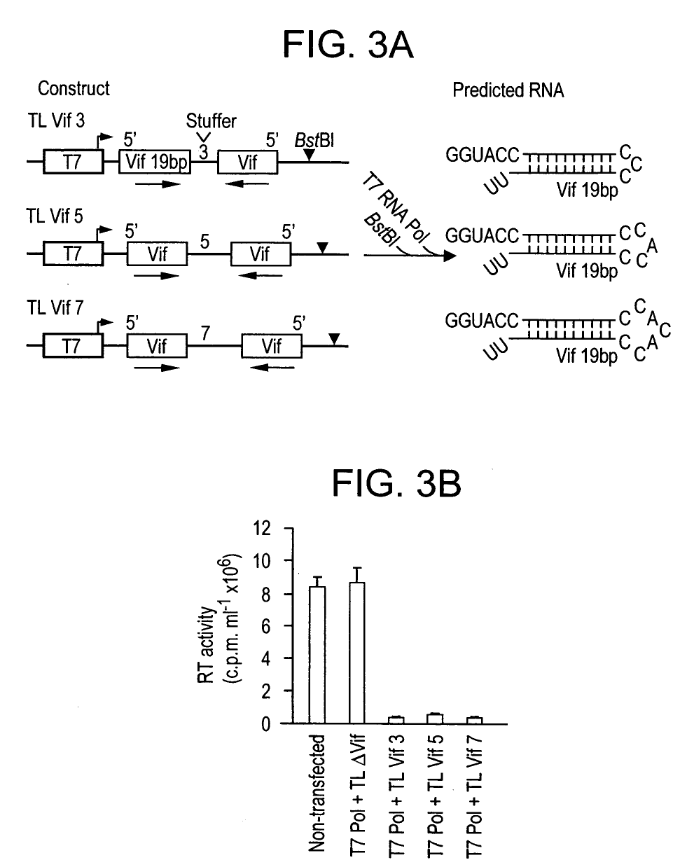

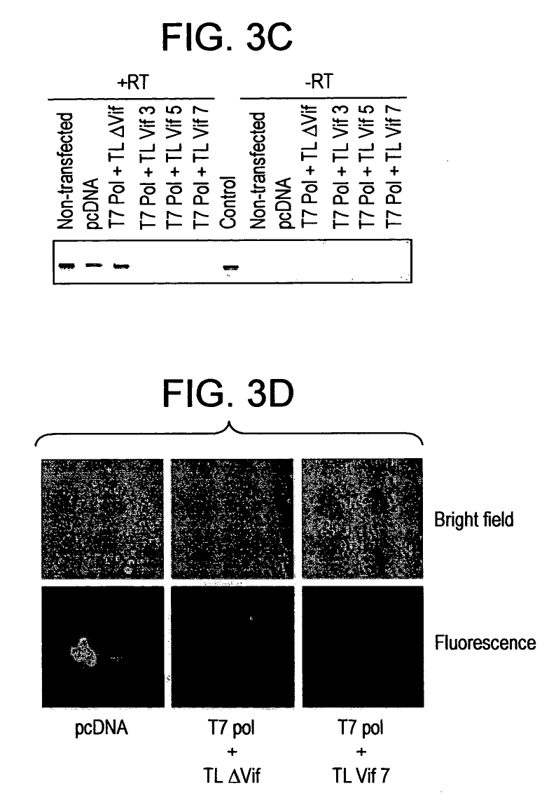

The term “small interfering RNA” (“siRNA”), also referred to in the art as “short interfering RNAs,” refers to an RNA (or RNA analog) comprising between about 10-50 nucleotides (or nucleotide analogs) which is capable of directing or mediating RNA interference. In preferred embodiments, an siRNA comprises about 15-30 nucleotides (or nucleotide analogs), 20-25 nucleotides (or nucleotide analogs), or 21-23 nucleotides (or nucleotide analogs). Unless otherwise indicated herein, the term “siRNA” refers to double stranded siRNA (as compared to single stranded or antisense RNA). The term “short hairpin RNA” (“shRNA”) refers to an siRNA (or siRNA analog) which is folded into a hairpin structure. shRNAs typically comprise about 45-60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. Exemplary shRNAs are depicted in FIG. 3A and discussed in the examples.

A siRNA having a “sequence sufficiently complementary to a portion of the HIV genome to mediate RNA interference (RNAi)” means that the siRNA has a sequence sufficient to trigger the destruction of the target RNA by the RNAi machinery or process. A completely complementary siRNA contains no mismatches as compared to the target RNA, e.g., a portion of the single-stranded RNA of the HIV genome. The siRNAs can include siRNA analogs that have one or more altered or modified nucleotides, or nucleotide analogs, as compared to a corresponding completely complementary siRNA, but retains the same or similar nature or function as the corresponding unaltered or unmodified siRNA. Such alterations or modifications can further include addition of non-nucleotide material, e.g., at one or both the ends of the siRNA or internally (at one or more nucleotides of the siRNA). An siRNA analog need only be sufficiently similar to the target RNA (e.g., a portion of viral RNA or MRNA), such that it has the ability to mediate RNA interference. The term “siRNA complex” refers to a complex of siRNA and proteins that recognize and degrade RNAs with a sequence sufficiently homologous to that of the siRNA.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein “early stages of replication” means the stages of viral replication that occur prior to integration of the viral DNA into the host cell's chromosome, and “late stages of replication” means the stages of replication that occur after integration of the viral DNA into the host cell's chromosome. Events exemplifying late stages of replication include, but are not limited to, production of viral RNAs, translation of viral proteins, and release of virions.

As used herein “retrovirus” or “retroviruses” refers to any of a group of viruses that contain RNA and reverse transcriptase. Retroviruses include, but are not limited to HIV, HTLV, and Hepatitis, including types and subtypes, e.g., HIV-1, HIV-2, Hepatitis A, B, C, D or E, or HTLV-BLV.

Various aspects of the invention are described in further detail in the following subsections.

HIV Virus

The Human Immunodeficiency Virus (HIV), refers to a family of closely-related retroviruses that cause profound immune system dysfunction over time. Acquired Immune Deficiency Syndrome (AIDS) is primarily caused as a result of an immune system weakened by the HIV virus. HIV, outside a host cell (primarily cells that have the CD4 co-receptor protein, e.g., lymphocytes, T4-lymphocytes or T-cells, macrophages, monocytes and dendritic cells), exists as a single-stranded RNA genome. The HIV genome is packaged in a protein core and membrane envelope along with virus-encoded integrase and reverse transcriptase enzyme. Upon entry of the host cell, the viral RNA is converted to DNA by the reverse transcriptase enzyme that is capable of polymerizing DNA.

There are two major types of HIV, type 1 (HIV-1) and type 2 (HIV-2). There are also subtypes within each type. HIV is flanked by long terminal repeat (LTR) regions. The viral genome includes genes that encode for: the major structural proteins, gag, pol (codes for enzymes generated by the virus such as reverse transcriptase, integrase and protease), and env (codes for CD4 receptor binding protein); the regulatory proteins, tat (codes for transactivation protein), and rev; and accessory proteins, vpu (involved in virion release and mechanism for CD4 degradation), vpr, vif (viral infectivity factor), and nef (involved in the downregulation of CD4 cell-surface expression, the activation of T cells, and the stimulation of HIV infectivity).

The replication cycle of HIV is well known, and can be generally characterized as follows. First, the virus enters the host cell either by fusion with the cell membrane at the surface of the cell, or by endocytosis. Once inside the cell, the viral envelope and capsid are lost, and the pre-integration complex (HIV genome and virus-encoded reverse transciptase enzyme) by integrase produce a viral cDNA. The viral cDNA is then integrated into the host cell's chromosome: HIV cDNA enters the host cell nucleus and the enzyme integrase inserts it into the host cell's DNA. Once the HIV DNA is inserted into the host cell's DNA, it is referred to as a provirus. The host cell machinery is then utilized to transcribe copies of the viral RNA that will be assembled into a new virus or translated into proteins that become part of the viral particle or regulate its assembly and the budding process. Accordingly, viral RNA is translated into viral reverse transcriptase, and envelope and structural proteins, and these components are assembled at the host cell wall to manufacture mature HIV virions that are subsequently released from the host cell. Some of the viral proteins require protease enzyme (also coded by the viral cDNA) for processing.

siRNA Molecules

The present invention features siRNA molecules, methods of making siRNA molecules and methods (e.g., research and/or therapeutic methods) for using siRNA molecules. The siRNA molecule can have a length from about 10-50 or more nucleotides (or nucleotide analogs), about 15-25 nucleotides (or nucleotide analogs), or about 20-23 nucleotides (or nucleotide analogs). The siRNA molecule can have nucleotide (or nucleotide analog) lengths of about 10-20, 20-30, 30-40, 40-50, 15, 16, 17, 18, 19, 20, 21, 22, 23, 24, 25, 26, 27, or 28. In a preferred embodiment, the siRNA molecule has a length of 21 nucleotides. It is to be understood that all ranges and values encompassed in the above ranges are within the scope of the present invention. Long dsRNAs to date generally are less preferable as they have been found to induce cell self-destruction known as interferon response in human cells. siRNAs can preferably include 5′ terminal phosphate and a 3′ short overhangs of about 2 nucleotides. In a preferred embodiment, the siRNA can be a short hairpin siRNA (shRNA). Even more preferably, the shRNA is an expressed shRNA. Examples of such shRNAs and methods of manufacturing the same are discussed in the examples. In another embodiment, the siRNA can be associated with one or more proteins in an siRNA complex.

The siRNA molecules of the invention include a sequence that is sequence sufficiently complementary to a portion of the viral (e.g., HIV, HTLV, and Hepatitis) genome to mediate RNA interference (RNAi), as defined herein, i e., the siRNA has a sequence sufficiently specific to trigger the degradation of the target RNA by the RNAi machinery or process. The siRNA molecule can be designed such that every residue of the antisense strand is complementary to a residue in the target molecule. Alternatively, substitutions can be made within the molecule to increase stability and/or enhance processing activity of said molecule. Substitutions can be made within the strand or can be made to residues at the ends of the strand.

The target RNA cleavage reaction guided by siRNAs is highly sequence specific. In general, siRNA containing a nucleotide sequences identical to a portion of the target gene are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition as shown in the examples. Alternatively, siRNA sequences with nucleotide analog substitutions or insertions can be effective for inhibition.

Moreover, not all positions of a siRNA contribute equally to target recognition. Mismatches in the center of the siRNA are most critical and can essentially abolish target RNA cleavage. In contrast, the 3′ nucleotides of the siRNA typically do not contribute significantly to specificity of the target recognition. In particular, 3' residues of the siRNA sequence which are complementary to the target RNA (e.g., the guide sequence) generally are not critical for target RNA cleavage.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=# of identical positions/total # of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin & Altschul, Proc. Natl. Acad. Sci. USA 87:2264-68 (1990), modified as in Karlin & Altschul, Proc. Natl. Acad. Sci. USA 90:5873-77 (1993). Such an algorithm is incorporated into the BLAST programs (version 2.0) of Altschul, et al., J. Mol. Biol. 215:403-10 (1990).

In another embodiment, the alignment is optimized by introducing appropriate gaps and percent identity is determined over the length of the aligned sequences (i. e., a gapped alignment). To obtain gapped alignments for comparison purposes, Gapped BLAST can be utilized as described in Altschul, et al., Nucleic Acids Res. 25(17):3389-3402 (1997). In another embodiment, the alignment is optimized by introducing appropriate gaps and percent identity is determined over the entire length of the sequences aligned (i.e., a global alignment). A so preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Greater than 90% sequence identity, e.g., 91%, 92%, 93%, 94%, 95%, 96%, 97%, 98%, 99% or even 100% sequence identity, between the siRNA and the portion of the target gene is preferred. In the context of an siRNA of about 20-25 nucleotides, e.g., at least 16-21 identical nucleotides are preferred, more preferably at least 17-22 identical nucleotides, and even more preferably at least 18-23 or 19-24 identical nucleotides. Alternatively worded, in an siRNA of about 20-25 nucleotides in length, siRNAs having no greater than about 4 mismatches are preferred, preferably no greater than 3 mismatches, more preferably no greater than 2 mismatches, and even more preferably no greater than 1 mismatch.

Alternatively, the siRNA may be defined functionally as a nucleotide sequence (or oligonucleotide sequence) that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log10[Na+])+0.41(% G+C) (600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., et al., 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel, et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

In one embodiment, the RNA molecules of the present invention are modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference. For example, the absence of a 2′ hydroxyl may significantly enhance the nuclease resistance of the siRNAs in tissue culture medium.

In an especially preferred embodiment of the present invention the RNA molecule may contain at least one modified nucleotide analogue. The nucleotide analogues may be located at positions where the target-specific activity, e.g., the RNAi mediating activity is not substantially effected, e.g., in a region at the 5′-end and/or the 3′-end of the RNA molecule. Particularly, the ends may be stabilized by incorporating modified nucleotide analogues.

Preferred nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides the phosphoester group connecting to adjacent ribonucleotides is replaced by a modified group, e.g., of phosphothioate group. In preferred sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In some embodiments, the siRNA can be modified by the substitution of at least one nucleotide with a modified nucleotide. The siRNA can have one or more mismatches when compared to the target sequence of the HIV genome and still mediate RNAi as demonstrated in the examples below.

The ability of the siRNAs of the present invention to mediate RNAi is particularly advantageous considering the rapid mutation rate of the HIV virus. The invention contemplates several embodiments which further leverage this ability by, e.g., targeting conserved regions of the HIV genome, synthesizing patient-specific siRNAs or plasmids, and/or introducing several siRNAs staggered along the HIV genome. In one embodiment, highly and/or moderately conserved regions of the HIV genome are targeted as discussed in greater detail below. Additionally or alternatively, a subject's infected cells can be procured and the genome of the HIV virus within it sequenced or otherwise analyzed to synthesize one or more corresponding siRNAs, plasmids or transgenes. Additionally or alternatively, high mutation rates can be addressed by introducing several siRNAs that target different and/or staggered regions of the HIV genome.

Manufacture of siRNA

In one embodiment, siRNAs are synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the siRNA. Inhibition may be targeted by specific transcription in an organ, tissue, or cell type; stimulation of an environmental condition (e.g., infection, stress, temperature, chemical inducers); and/or engineering transcription at a developmental stage or age. A transgenic organism that expresses siRNA from a recombinant construct may be produced by introducing the construct into a zygote, an embryonic stem cell, or another multipotent cell derived from the appropriate organism.

In addition, not only can an siRNA be used to cleave multiple RNAs within the cell, but the siRNAs can be replicated and amplified within a cell by the host cell enzymes. Alberts, et al., The Cell 452 (4th Ed. 2002). Thus, a cell and its progeny can continue to carry out RNAi even after the HIV RNA has been degraded.

RNA may be produced enzymatically or by partial/total organic synthesis, any modified ribonucleotide can be introduced by in vitro enzymatic or organic synthesis. In one embodiment, a siRNA is prepared chemically. Methods of synthesizing RNA molecules are known in the art, in particular, the chemical synthesis methods as de scribed in Verma and Eckstein, Annul Rev. Biochem. 67:99-134 (1998). In another embodiment, a siRNA is prepared enzymatically. For example, a siRNA can be prepared by enzymatic processing of a long dsRNA having sufficient complementarity to the desired target RNA. Processing of long dsRNA can be accomplished in vitro, for example, using appropriate cellular lysates and ds-siRNAs can be subsequently purified by gel electrophoresis or gel filtration. In an exemplary embodiment, RNA can be purified from a mixture by extraction with a solvent or resin, precipitation, electrophoresis, chromatography, or a combination thereof. Alternatively, the RNA may be used with no or a minimum of purification to avoid losses due to sample processing.

The siRNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan & Uhlenbeck, Methods Enzymol. 180:51-62 (1989)). The RNA may be dried for storage or dissolved in an aqueous solution. The solution may contain buffers or salts to inhibit annealing, and/or promote stabilization of the single strands.

siRNA Vectors

Another aspect of the present invention includes a vector that expresses one or more siRNAs that include sequences sufficiently complementary to a portion of the HIV genome to mediate RNAi. The vector can be administered in vivo to thereby initiate RNAi therapeutically or prophylactically by expression of one or more copies of the siRNAs.

In one embodiment, synthetic shRNA is expressed in a plasmid vector. In another, the plasmid is replicated in vivo. In another embodiment, the vector can be a viral vector, e.g., a retroviral vector. Examples of such plasmids and methods of making the same are illustrated in the examples. Use of vectors and plasmids are advantageous because the vectors can be more stable than synthetic siRNAs and thus effect long-term expression of the siRNAs.

The HIV genome mutates rapidly and a mismatch of even one nucleotide can, in some instances, impede RNAi. Accordingly, in one embodiment, a vector is contemplated that expresses a plurality of siRNAs to increase the probability of sufficient homology to mediate RNAi. Preferably, these siRNAs are staggered along the HIV genome. In one embodiment, one or more of the siRNAs expressed by the vector is a shRNA. The siRNAs can be staggered along one portion of the HIV genome or target different genes in the HIV genome. In one embodiment, the vector encodes about 3 siRNAs, more preferably about 5 siRNAS. The siRNAs can be targeted to conserved regions of the HIV genome, e.g., the vif region and/or the regions coding for reverse transcriptase and/or protease. Additionally or alternatively, the siRNAs can be targeted to the rev or vif region of the HIV genome. Additionally, or alternatively, the siRNAs can be targeted to the gag region, the vpr region, and/or one or more regions coding for envelope proteins, structural or core proteins and/or the LTR region.

Long dsRNAs

The involvement of RNAi in transposon silencing (Ketting, R. F., et al., Cell 99, 133-141 (1999); Tabara, H., et al., Development 126, 1-11 (1999)) suggests that RNAi is an ancient antiviral system that may have evolved as a defense mechanism to protect the host from invasion by mobile genetic elements including transposons and viruses. Several studies have indicated that it is difficult to induce RNAi in mammalian cells using long dsRNAs. Although long dsRNAs can inhibit gene expression in mammalian cells, the effects are not sequence specific (Elbashir, S. M., et al., Nature 411, 494-498 (2001); Caplen, N. J., et al., Proc. Natl Acad. Sci. USA 98, 9742-9747 (2001) and are more consistent with inhibition by the interferon response. Intriguingly, it is now becoming apparent that underlying the non-specific dsRNA-activated interferon response in mammalian cells, there may indeed be a sequence-specific RNAi effect that can be activated by long dsRNA (Billy, E., et al., Proc. Natl Acad. Sci. USA 98, 14428-14433 (2001); Paddison, P. J., et al., Proc. Natl Acad. Sci. USA 99, 1443-1448 (2002); Yang, S., et al., Cell Biol. 21, 7807-7816 (2001). Silencing by long dsRNAs has now been observed in various cultured mammalian cells (Billy, E., et al., Proc. Natl Acad. Sci. USA 98, 14428-14433 (2001); Paddison, P. J., et al., Proc. Natl Acad. Sci. USA 99, 1443-1448 (2002). The mechanism of silencing is consistent with RNAi because there is evidence that the long dsRNAs are processed to siRNAs and target RNAs are specifically degraded. The results presented herein indicate that 21-nucleotide siRNAs promote HIV RNA degradation in primary lymphocytes, suggesting that the major target cell for HIV replication possesses functional components of the siRNA-induced silencing complex that mediates specific cleavage of target RNA (Hutvagner, G. & Zamiore, P. D., Curr. Opin. Genet. Dev. 12, 225-232 (2002). It follows that sequence-specific RNAi that is independent of the interferon response can be activated against HIV by long dsRNAs.

HIV Genome Targets

In one embodiment, the siRNA inhibits the synthesis of viral HIV cDNA. In another, the siRNA promotes the degradation of or inhibits synthesis of viral HIV cDNA intermediates. In yet another, the siRNA promotes the degradation of or inhibits synthesis of viral HIV RNA. The siRNA can mediate RNAi during an early viral replication cycle event and/or a late viral replication cycle event.

Target portions of the HIV genome include, but are not limited to, the Long Terminal Repeats (LTR) of the HIV genome, the nef gene, or the vif gene. The target portion of the HIV genome can be the portion of the genomic RNA that specifies the amino acid sequence of a viral HIV protein or enzyme (e.g., a reverse transcriptase enzyme, a capsid protein or envelope protein). As used herein, the phrase “specifies the amino acid sequence” of a protein means that the RNA sequence is translated into the amino acid sequence according to the rules of the genetic code. The protein may be a viral protein involved in immunosuppression of the host, replication of HIV, transmission of the HIV, or maintenance of the infection.

In one embodiment, the target portion of the HIV genome is a highly conserved region. In another embodiment, HIV virus is extracted from a patient and the siRNA is produced to match a portion of the HIV genome that has mutated. This can be done for generations of HIV mutations to mediate RNAi in a patient that develops resistance to previously used siRNAs.

In embodiments where a series of siRNAs are introduced to a cell or organism, preferably the series of siRNAs correspond to one or more highly conserved region of the HIV genome. When targeting highly conserved regions, relatively few siRNAs can be effective in mediating RNAi despite mutations in the genome. Highly conserved regions include the pol region encoding, e.g., for protease and reverse transcriptase, and the tat, rev, and vif genes. In a preferred embodiment, at least 3 siRNAs are expressed corresponding to the portion of the pol region that encodes protease and/or reverse transcriptase enzyme, and/or the vif region. In another embodiment at least 5 siRNAs are expressed corresponding to the regions of the HIV genome encoding protease and/or reverse transcriptase, and/or tat, rev, and/or vif genes. The siRNAs can also correspond to the LTR regions, the gag gene, the vpr gene, and/or the env gene.

Methods of Introducing RNAs, Vectors, and Host Cells

Physical methods of introducing the agents of the present invention (e.g., siRNAs, vectors, or transgenes) include injection of a solution containing the agent, bombardment by particles covered by the agent, soaking the cell or organism in a solution of the agent, or electroporation of cell membranes in the presence of the agent. A viral construct packaged into a viral particle would accomplish both efficient introduction of an expression construct into the cell and transcription of RNA, including siRNAs, encoded by the expression construct. Other methods known in the art for introducing nucleic acids to cells may be used, such as lipid-mediated carrier transport, chemical-mediated transport, such as calcium phosphate, and the like. Thus the siRNA may be introduced along with components that perform one or more of the following activities: enhance siRNA uptake by the cell, inhibit annealing of single strands, stabilize the single strands, or otherwise increase inhibition of the target gene.

The agents may be directly introduced into the cell (i e., intracellularly); or introduced extracellularly into a cavity, interstitial space, into the circulation of an organism, introduced orally, or may be introduced by bathing a cell or organism in a solution containing the RNA. Vascular or extravascular circulation, the blood or lymph system, and the cerebrospinal fluid are sites where the agent may be introduced.

Cells may be infected with HIV upon delivery of the agent or exposed to the HIV virus after delivery of agent. The cells may be derived from or contained in any organism. The cell may be from the germ line, somatic, totipotent or pluripotent, dividing or non-dividing, parenchyma or epithelium, immortalized or transformed, or the like. The cell may be a stem cell, e.g., a hematopoietic stem cell, or a differentiated cell. Cell types that are differentiated include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. Preferably, the cell is a lymphocyte (such as a T lymphocyte), a macrophage (such as a monocytic macrophage), a monocyte, or is a precursor to either of these cells, such as a hematopoietic stem cell. In a preferred embodiment, the cell is a primary peripheral lymphocyte.

Depending on the particular target gene and the dose of double stranded RNA material delivered, this process may provide partial or complete loss of function for the target gene. A reduction or loss of gene expression in at least 50%, 60%, 70%, 80%, 90%, 95% or 99% or more of targeted cells is exemplary. Inhibition of gene expression refers to the absence (or observable decrease) in the level of viral protein, RNA, and/or DNA. Specificity refers to the ability to inhibit the target gene without manifesting effects on other genes, particularly those of the host cell. The consequences of inhibition can be confirmed by examination of the outward properties of the cell or organism or by biochemical techniques such as RNA solution hybridization, nuclease protection, Northern hybridization, reverse transcription gene expression monitoring with a microarray, antibody binding, enzyme linked immunosorbent assay (ELISA), integration assay, Western blotting, radioimmunoassay (RIA), other immunoassays, and fluorescence activated cell analysis (FACS).

For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentarnycin, hygromycin, kanamycin, lincomycin, methotrexate, phosphinothricin, puromycin, and tetracyclin. Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of siRNA may result in inhibition in a smaller fraction of cells (e.g., at least 10%, 20%, 50%, 75%, 90%, or 95% of targeted cells).

Quantitation of gene expression in a cell may show similar amounts of inhibition at the level of accumulation of target RNA or translation of target protein. As an example, the efficiency of inhibition may be determined by assessing the amount of gene product in the cell; RNA may be detected with a hybridization probe having a nucleotide sequence outside the region used for the inhibitory double-stranded RNA, or translated polypeptide may be detected with an antibody raised against the polypeptide sequence of that region.

The siRNA may be introduced in an amount that allows delivery of at least one copy per cell. Higher doses (e.g., at least 5, 10, 100, 500 or 1000 copies per cell) of material may yield more effective inhibition; lower doses may also be useful for specific applications.

Methods of Treatment

The present invention provides for both prophylactic and therapeutic methods for treating a subject at risk of (or susceptible to) or a subject having a virus (e.g., HIV virus, Human T-cell Lukemia Virus, and viral Hepatitis). “Treatment”, or “treating” as used herein, is defined as the application or administration of a therapeutic agent (e.g., a siRNA or vector or transgene encoding same) to a patient, or application or administration of a therapeutic agent to an isolated tissue or cell line from a patient, who has a virus with the purpose to cure, heal, alleviate, relieve, alter, remedy, ameliorate, improve or affect the virus, or symptoms of the virus. The term “treatment” or “treating” is also used herein in the context of administering agents prophylactically, e.g., to inoculate against a virus.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, infection with the HIV virus or a condition associated with the HIV virus, e.g., AIDS, by administering to the subject a prophylactically effective agent that includes any of the siRNAs or vectors or transgenes discussed herein. Administration of a prophylactic agent can occur prior to the manifestation of symptoms characteristic of HIV infection, such that HIV infection, AIDS and/or AIDS related diseases are prevented.

In a preferred embodiment, the prophylactically effective agent is administered to the subject prior to exposure to the HIV virus to prevent its integration into the host's cells. In another embodiment, the agent is administered to the subject after exposure to the HIV virus to delay or inhibit its progression, or prevent its integration into the DNA of healthy cells or cells that do not contain a provirus. Thus, the method is prophylactic in the sense that healthy cells are protected from HIV infection. The methods generally include administering the agent to the subject such that HIV replication or infection is prevented or inhibited. Preferably, HIV provirus formation is inhibited or prevented. Additionally or alternatively, it is preferable that HIV replication is inhibited or prevented. In one embodiment, the siRNA degrades the HIV RNA in the early stages of its replication, for example, immediately upon entry into the cell. In this manner, the agent can prevent healthy cells in a subject from becoming infected. In another embodiment, the siRNA degrades the viral MRNA in the late stages of replication. Any of the strategies discussed herein can be employed in these methods, such as administration of a vector that expresses a plurality of siRNAs sufficiently complementary to the HIV genome to mediate RNAi.

2. Therapeutic Methods

Another aspect of the invention pertains to methods of modulating target gene expression, protein expression or activity for therapeutic purposes. Accordingly, in an exemplary embodiment, the modulatory method of the invention involves contacting a cell infected with the virus with a therapeutic agent (e.g., a siRNA or vector or transgene encoding same) that is specific for the a portion of the viral genome such that RNAi is mediated. These modulatory methods can be performed ex vivo (e.g., by culturing the cell with the agent) or, alternatively, in vivo (e.g., by administering the agent to a subject). The methods can be performed ex vivo and then the products introduced to a subject (e.g., gene therapy).

The therapeutic methods of the invention generally include initiating RNAi by administering the agent to a subject infected with the virus (e.g., HIV, HTLV, and Hepatitis). The agent can include one or more siRNAs, one or more siRNA complexes, vectors that express one or more siRNAs (including shRNAs), or transgenes that encode one or more siRNAs. The therapeutic methods of the invention are capable of reducing viral production (e.g., viral titer or provirus titer), by about 30-50-fold, preferably by about 60-80-fold, and more preferably about (or at least) 90-fold, 100-fold, 200-fold, 300-fold, 400-fold, 500-fold or 1000-fold.

In a preferred embodiment, infected cells are obtained from a subject and analyzed to determine one or more sequences from the virus genomes present in that subject, siRNA is then synthesized to be sufficiently homologous to mediate RNAi (or vectors are synthesized to express such siRNAs), and delivered to the subject. This approach is advantageous because it addresses the particular virus mutations present in the subject. This method can be repeated periodically, to address further mutations in that subject and/or provide boosters for that subject.

Additionally, the therapeutic agents and methods of the present invention can be used in co-therapy with post-transcriptional approaches (e.g., with ribozymes and/or antisense siRNAs).

3. Dual Prophvlactic and Therapeutic Method

In a preferred method, a two-pronged attack on the HIV virus is effected in a subject that has been exposed to the HIV virus. An infected subject can thus be treated both prophylactically and therapeutically, such that the agent prevents infection of non-proviral cells by degrading the virus during early stages of replication and prior to integration into the host cell genome, and also retards replication of the virus in cells in which the HIV has already integrated itself into the host cell genome.

One skilled in the art can readily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect (e.g., with surrogate indicators of viral infection, such as p24 or reverse transcriptase for treatment of AIDS or AIDS-like disease) analysis of appropriate patient samples (e.g., blood and/or tissues).

Further, with respect to determining the effective level in a patient for treatment of AIDS or AIDS-like disease, in particular, suitable animal models are available and have been widely implemented for evaluating the in vivo efficacy against HIV of various gene therapy protocols (Sarver, et al., AIDS Res. and Hum. Retrovir. 9: 483-487 (1993)). These models include mice, monkeys, and cats. Even though these animals are not naturally susceptible to HIV disease, chimeric mice models (e.g., SCID, bg/nu/xid, bone marrow-ablated BALB/c) reconstituted with human peripheral blood mononuclear cells (PBMCs), lymph nodes, or fetal liver/thymus tissues can be infected with HIV, and employed as models for HIV pathogenesis and gene therapy. Similarly, the simian immune deficiency virus (SIV)/monkey model can be employed, as can the feline immune deficiency virus (FIV)/cat model. Mice expressing siRNAs against hepatitis C RNA have demonstrated that siRNAs can work in a living mammal to prevent viral replication (McCaffrey, et al., Nature 418:38-39 (2002)). For example, to induce a patient to manufacture siRNA, the patient's cells (e.g., bone marrow cells), can be transfected with siRNA genes and reintroduced into the patient's body.

The prophylactic or therapeutic pharmaceutical compositions of the present invention can contain other pharmaceuticals, in conjunction with a vector according to the invention, when used to therapeutically treat AIDS. These other pharmaceuticals can be used in their traditional fashion (i.e., as agents to treat HIV infection), as well as more particularly, in the method of selecting for conditionally replicating HIV (crHIV) viruses in vivo. Such selection as described herein will promote crHIV spread, and allow crHIV to more effectively compete with wild-type HIV, which will necessarily limit wild-type HIV pathogenicity. In particular, it is contemplated that an antiretroviral agent be employed, such as, for example, zidovudine. Further representative examples of these additional pharmaceuticals that can be used in addition to those previously described, include antiviral compounds, immunomodulators, immunostimulants, antibiotics, and other agents and treatment regimes (including those recognized as alternative medicine) that can be employed to treat AIDS. Antiviral compounds include, but are not limited to, ddI, ddC, gancylclovir, fluorinated dideoxynucleotides, nonnucleoside analog compounds such as nevirapine (Shih, et al., PNAS 88: 9978-9882 (1991)), TIBO derivatives such as R82913 (White, et al., Antiviral Research 16: 257-266 (1991)), and BI-RJ-70 (Shih, et al., Am. J. Med. 90 (Suppl. 4A): 8S-17S (1991)). Immunomodulators and immunostimulants include, but are not limited to, various interleukins, CD4, cytokines, antibody preparations, blood transfusions, and cell transfusions. Antibiotics include, but are not limited to, antifungal agents, antibacterial agents, and anti-Pneumocystis carinii agents.

Administration of siRNAs or vectors with other anti-retroviral agents and particularly with known RT inhibitors, such as ddC, zidovudine, ddI, ddA, or other inhibitors that act against other HIV proteins, such as anti-TAT agents, can be used to inhibit most or all replicative stages of the viral life cycle. The dosages of ddC and zidovudine used in AIDS or ARC patients have been published. A virustatic range of ddC is generally between 0.05 μM to 1.0 μM. A range of about 0.005-0.25 mg/kg body weight is virustaic in most patients. The dose ranges for oral administration are somewhat broader, for example, 0.001 to 0.25 mg/kg given in one or more doses at intervals of 2, 4, 6, 8, and 12 hours. Preferably, 0.01 mg/kg body weight ddC is given every 8 hours. When given in combined therapy, the other antiviral compound, e.g., can be given at the same time as a vector according to the invention, or the dosing can be staggered as desired. The vector also can be combined in a composition. Doses of each can be less, when used in combination, than when either is used alone.

A siRNA or vector according to the invention can be delivered to cells cultured ex vivo prior to reinfusion of the transfected cells into the patient or in a delivery vehicle complex by direct in vivo injection into the patient or in a body area rich in the target cells. The in vivo injection may be made subcutaneously, intravenously, intramuscularly or intraperitoneally. Techniques for ex vivo and in vivo gene therapy are known to those skilled in the art. Generally, the compositions are administered in a manner compatible with the dosage formulation, and in such amount as will be prophylactically and/or therapeutically effective. The quantity to be administered depends on the subject to be treated, including, e.g., whether the subject has been exposed to HIV or infected with HIV, or is afflicted with AIDS, and the degree of protection desired. Suitable regimens for initial administration and booster shots are also variable but are typified by an initial administration followed by subsequent inoculations or other administrations. Precise amounts of active ingredients required to be administered depend on the judgment of the practitioner and may be peculiar to each subject. It will be apparent to those of skill in the art that the therapeutically effective amount of a composition of this invention will depend upon the administration schedule, the unit dose of agent (e.g., siRNA, vector and/or transgene) administered or expressed by an expression plasmid that is administered, whether the compositions are administered in combination with other therapeutic agents, the immune status and health of the recipient, and the therapeutic activity of the particular nucleic acid molecule, delivery complex, or ex vivo transfected cell.

As such, the present invention provides methods of treating an individual afflicted with HIV.

4. Pharmacogenomics

The prophylactic and/or therapeutic agents (e.g., a siRNA or vector or transgene encoding same) of the invention can be administered to treat (prophylactically or therapeutically) individuals infected with a virus such as retrovirus (e.g., HIV, HTLV, and Hepatitis). In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a therapeutic agent as well as tailoring the dosage and/or therapeutic regimen of treatment with a therapeutic agent.

Pharmacogenomics deals with clinically significant hereditary variations in the response to drugs due to altered drug disposition and abnormal action in affected persons. See, for example, Eichelbaum, M., et al., Clin. Exp. Pharmacol. Physiol. 23(10-11): 983-985 (1996) and Linder, M. W., et al., Clin. Chem. 43(2):254-266 (1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict drug response. According to this method, if a gene that encodes a drugs target is known (e.g., a target gene polypeptide of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling”, can be utilized to identify genes that predict drug response. For example, the gene expression of an animal dosed with a therapeutic agent of the present invention can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with a therapeutic agent, as described herein.

Therapeutic agents can be tested in an appropriate animal model. For example, a siRNA (or expression vector or transgene encoding same) as described herein can be used in an animal model to determine the efficacy, toxicity, or side effects of treatment with said agent. Alternatively, a therapeutic agent can be used in an animal model to determine the mechanism of action of such an agent. For example, an agent can be used in an animal model to determine the efficacy, toxicity, or side effects of treatment with such an agent. Alternatively, an agent can be used in an animal model to determine the mechanism of action of such an agent.

Pharmaceutical Compositions

The invention pertains to uses of the above-described agents for the prophylactic and therapeutic treatments as described infra. Accordingly, the agents of the present invention can be incorporated into pharmaceutical compositions suitable for administration. Such compositions typically comprise the agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral (e.g., intravenous, intradermal, subcutaneous, intraperitoneal, and intramuscular), oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fingi. The carrier can be a solvent or dispersion medium containing, for example, water, ethanol, polyol (e.g., glycerol, propylene glycol, and liquid polyetheylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal agents (e.g., parabens, chlorobutanol, phenol, ascorbic acid, thimerosal, and the like). In many cases, it will be preferable to include isotonic agents (e.g., sugars, polyalcohols such as manitol, sorbitol, and sodium chloride) in the composition. Prolonged absorption of the injectable compositions can be brought about by including in the composition an agent that delays absorption (e.g., aluminum monostearate and gelatin).

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of so tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

The pharmaceutical compositions can be included in a container, pack, or dispenser together with instructions for administration.

Knockout and/or Knockdown Cells or Organisms

A further preferred use for the siRNAs of the present invention (or vectors or transgenes encoding that subsequently express siRNAs in the cell) is a functional analysis to be carried out in HIV eukaryotic cells, or eukaryotic non-human organisms, preferably mammalian cells or organisms and more preferably human cells, e.g. cell lines such as HeLa or 293 or rodents, e.g. rats and mice. In one embodiment, the cell is a lymphocyte or lymphocyte precursor, and more preferably a primary peripheral blood lymphocyte or its precursor. The cells may be infected with HIV virus or subsequently infected. The cell can include less than 500 copies, or less than 1000 copies of viral HIV RNA. The siRNAs, vectors or transgenes can be any of the agents discussed herein, e.g., a vector that expresses a plurality of shRNAs that target different portions of the HIV genome.

By administering a suitable siRNA molecule or molecules which are sufficiently homologous to a target portion of the HIV genome to mediate RNA interference, a specific knockout or knockdown phenotype can be obtained in a target cell, e.g. in cell culture or in a target organism.

Gene-specific knockout or knockdown phenotypes of cells or non-human organisms, particularly of human cells or non-human mammals may be used in analytic to procedures, e.g., in the functional and/or phenotypical analysis of complex physiological processes such as analysis of gene expression profiles and/or proteomes. Preferably the analysis is carried out by high throughput methods using oligonucleotide based chips.

This invention is further illustrated by the following examples that should not be construed as limiting. The contents of all references, patents and published patent applications cited throughout this application are incorporated herein by reference.

### EXAMPLES

HIV-1 uses RNA intermediates in its replication. Therefore, whether siRNA duplexes, specific for HIV-1, were capable of effecting the degradation of viral RNAs necessary for completion of early and late events in the viral replication cycle was examined.

Methodology

The following methodology was used in connection with the examples. Obvious variants will occur to the practitioner.

Synthesis of siRNA

The following RNA oligonucleotides were purchased from Dharmacon:

| | | |
|---|---|---|
| T98 | (5′-GGAAAGCUAAGGACUGGUUhndT | (SEQ ID NO: 1) |
| | dT-3′); | |
| T283 | (5′-AGCACACAAGUAGACCCUGdTd | (SEQ ID NO: 2) |
| | T-3′); | |
| T441: | 5′-CUUGGCACUAGCAGCAUUAdTdT- | (SEQ ID NO: 3) |
| | 3′); | |
| M98 | (5′ GAAAGCUAGGGGAUGGUUdTdT- | (SEQ ID NO: 4) |
| | 3′); | |
| M441 | (5′-CUUGGCACUAACAGCAUUAdTd | (SEQ ID NO: 5) |
| | T-3′); | |
| G388 | (5′-GACUUCAAGGAAGAUGGCAdTd | (SEQ ID NO: 6) |
| | T-3′); | |
| M388 | (5′-GACUUCAAGGGAGAUGGCAdTd | (SEQ ID NO: 7) |
| | T-3′); | |
| nef | (5′-GUGCCUGGCUAGAAGCACAdTd | (SEQ ID NO: 8) |
| | T-3′); | |
| TAR | (5′-AGACCAGAUCUGAGCCUGGdTd | (SEQ ID NO: 9) |
| and | T-3′); | |
| MTAR | (5′-AGACCAGAUAUGAGCCUGGdTd | (SEQ ID NO: 10) |
| | T-3′). | |

Plasmids

The T7 promoter was modified in the plasmid PCRscript (Stratagene) to form pCRT7. Oligonucleotides corresponding to nucleotides 5,323-5,342 of HIV-1 vif (Genbank accession number M19921) were inserted at the SrfI site of pCRT7. T7 pol comprises T7 RNA polymerase from Escherichia coli BL21 (DE3) cloned into pcDNA 3.1 (Invitrogen).

Cells and Transfections

Magi cells were grown in DMEM containing 10% fetal bovine serum (FBS). PHA-activated, elutriated PBLs were cultured in RPMI containing 10% FBS and 64 U ml⁻¹ of interleukin-2 (ICN). Magi cells were transfected with oligofectamine (GIBCO) by the manufacturer's protocol in the presence of 1 μg HIV plasmid and/or 60 pmol of siRNA oligonucleotides. Transfection efficiencies were 75-85%. For PHA-activated PBLs, 5×10⁶ cells were electroporated using a Gene Pulser apparatus (Bio-Rad) at 250 V, 960 μF, resistance R=∞ with 5 μg plasmid and/or 200 pmol siRNA. Transfection efficiencies were 30-50% of viable cells. Three-way transfections with siRNA expression plasmids comprised 0.1 μg T7 Pol, 0.5 μg pTL vif and 0.5 μg pNLGFP (Magi cells), or 0.5 μg T7 Pol, 2 μg TL vif and 2 μg pNLGFP (for primary lymphocytes). Transfected cells were centrifuged (1,200 g) on DAKO silanized slides and examined under bright-field illumination or fluorescence (wavelength 516 nm) on a Zeiss Axioplan 2 microscope.

PCR Analysis

Real-time PCR was performed as previously reported (Sharkey, M. et al., Nature Med. 6, 76-81 (2000)). Products were amplified from 5 to 20 μl of extrachromosomal DNA in 50-μl reactions containing 1×HotStart Taq buffer (Qiagen), 200 nM dNTPs, 400 nM primers and 1.5 U HotStart Taq. Two-LTR junctions were amplified by the primers Rc (5′-TAGACCAGATCTGAGCCTGGGA-3′)(SEQ ID NO: 11) and U5c (5′-GTAGTTCTGCCAATCAGGGAAG-3′)(SEQ ID NO: 12). Early products were amplified by the primers Ra (5′-TCTCTGGTTAGACCAGATCTG-3′)(SEQ ID NO: 12) and U5a (5′-GTCTGAGGGATCTCTAGTTAC-3′)(SEQ ID NO: 13), and late products were amplified with U5b (5′-GGGAGCTCTCTGGCTAACT-3′)(SEQ ID NO: 14) and gag (5′-GGATTAACTGCGAATCGTTC-3′) (SEQ ID NO: 15) primers. The oligonucleotide probe for real-time PCR was as previously reported (Sharkey, M., et al., Nature Med. 6, 76-81 (2000)).

Viral Assays

For RT-PCR, 1-2 μg RNA was reverse transcribed and amplified by PCR using the nef primers Na (5′-GACAGGGCTTGGAAAGG-3′) (SEQ ID NO: 16) and Nb (5′-TTAGCAGTTCTGAAGTACTC-3′) (SEQ ID NO: 17) as described previously (Brichacek, B. & Stevenson, M., Methods 12, 294-299 (1997). The integration assay was performed on DNAzol-extracted total DNA (Invitrogen) using the Alu primer SB704 (5′-TGCTGGGATTACAGGCGTGAG-3′) (SEQ ID NO: 18) and primer Rc for the first round of PCR (25 cycles). Nested PCR was performed under the same conditions using primers M667 (5′-GGCTAACTAGGGAACCCACTG-3′) (SEQ ID NO: 19) and AA55 (5′-CTGCTAGAGATTTTCCACACTGAC-3′) (SEQ ID NO: 20). For virus production, viral p24 (capsid) was measured by enzyme-linked immunosorbent assay according to the manufacturer's protocol (Beckman-Coulter). Reverse transcription activity was measured as previously reported (Brichacek, B. & Stevenson, M., Methods 12, 294-299 (1997)).

PKR Assays

20 μg of whole-cell lysates were electrophoresed in triple detergent lysis buffer on a 10% SDS-polyacrylamide gel and electrotransferred to a nitrocellulose membrane (Amersham Hybond C+). The membrane was probed with a phospho-Thr 446 PKR-specific antibody or a PKR-specific antibody (Upstate Biotechnology).

### Example I

### Reduction of HIV Virus Production with siRNAs with Completely Homologous siRNAs, and siRNAs with Mismatches

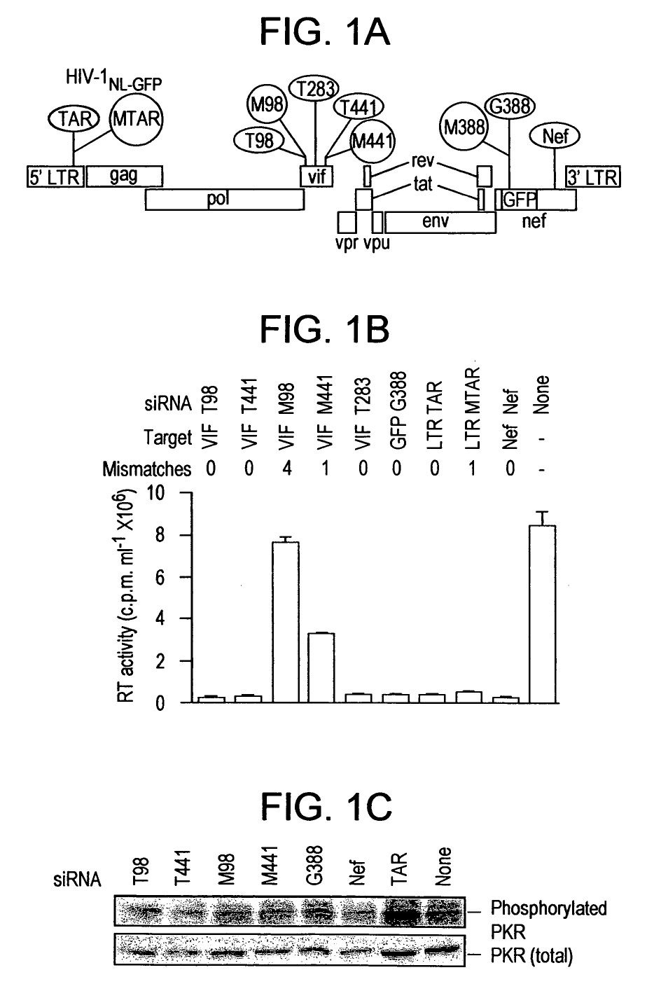

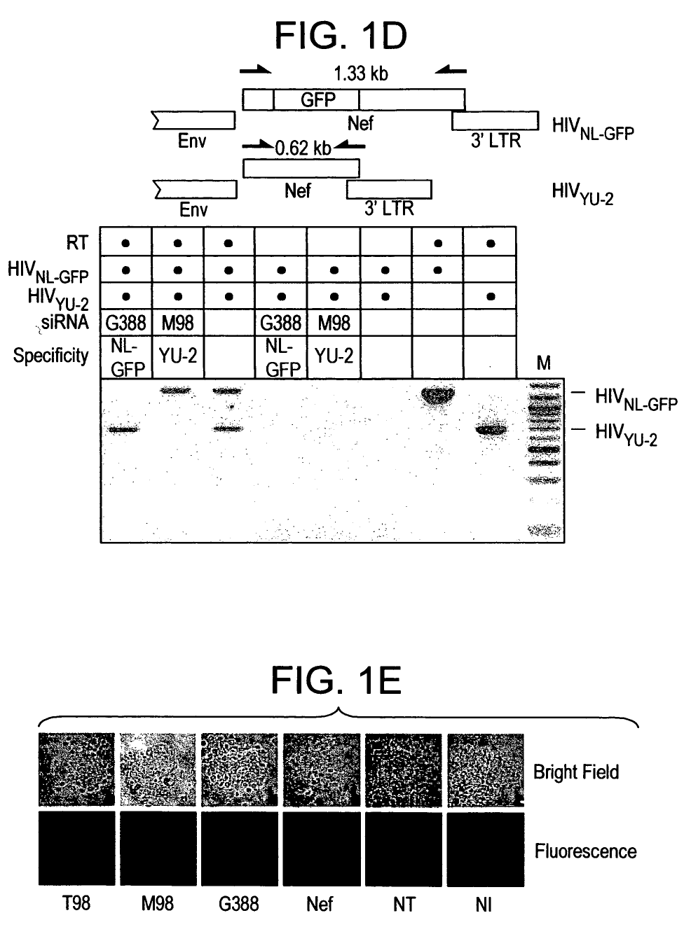

21-nucleotide siRNA duplexes were directed against several regions of the HIV-1 genome, including the viral long terminal repeat (LTR) and the accessory genes vif and nef (FIG. 1A). Small interfering RNA duplexes were co-transfected with an HIV-1 molecular clone (HIV_{NL-GFP}; Welker, R., et al., J. Virol. 72, 8833-8840 (1998) into CD4-positive HeLa (Magi) cells (Kimpton, J. & Emerman, M., J. Virol. 66, 2232-2239 (1992)). Transfection of cells with an infectious molecular HIV-1 clone recapitulates late events in the viral life cycle, including production of viral RNAs, translation of viral proteins and release of virions. Compared with cells not transfected with siRNA duplexes, virus production, measured 24 hours after transfection, was reduced 30-fold to 50-fold by homologous siRNAs (FIG. 1B). HIV production was inhibited to a lesser extent by single mismatch siRNAs (MTAR, M441), whereas a vif siRNA with four mismatches (M98) did not inhibit HIV production (FIG. 1B).

Example II

### siRNAs Inhibit HIV Production by Causing Sequence-specific Degradation of Viral RNA

Activation of the dsRNA-activated protein kinase PKR leads to an inhibition of protein translation in a sequence-non-specific manner relative to the inducing dsRNA. Activation with PKR was not involved in the inhibition of the negative-strand RNA virus RSV (respiratory syncytial virus) by siRNAs (Bitko, V. & Barik, S., BMC Microbiol. 1, 34-45 (2001)). Similarly, there was no significant induction of activated PKR (phosphorylated on Thr 446) over levels in non-transfected cells by any of the siRNAs (FIG. 1C). To further exclude a PKR effect, Magi cells were co-transfected with two HIV-1 variants (HIV-1_{NL-GFP}, HIV-1_{YU-2}; (Li, Y. et al., J. Virol., 65, 3973-3985 (1991)) and with siRNAs that are specifically targeted to either virus. Because of the presence of a green fluorescent protein (GFP) insertion in Nef, HIV_{NL-GFP} should be targeted by the GFP-specific siRNA G388, whereas HIV_{YU-2}, which lacks a GFP insert, should be insensitive to G388. In addition, sequence differences in the vif genes of these viruses were exploited. The M98 siRNA contains four mismatches relative to the HIVNL-GFP vif gene but is completely homologous to HIV_{YU-2} vif. Thus, M98 should direct the specific inhibition of HIV_{YU-2} RNA and not HIV_{NL-GFP} RNA. Because of the GFP insertion in HIV_{NL-GFP}, viral RNA produced in cells harboring both viruses could be distinguished. In the absence of siRNAs, both HIV_{NL-GFP} and HIV_{YU-2} RNAs were evident in co-transfected cells (FIG. 1D). However, co-transfection with the G388 siRNA resulted in a loss of HIV_{NL-GFP} RNA but not HIV_{YU-2} RNA. Conversely, the M98 siRNA caused a loss in HIV_{YU-2} RNA without affecting HIV_{NL-GFP} RNA (FIG. 1D). This sequence-specific inhibition is inconsistent with a sequence-non-specific PKR effect and indicates that siRNAs are inhibiting HIV production by causing the specific degradation of viral RNA.

### Example III

### Inhibition of HIV Expression in Lymphocytes

We next examined whether siRNAs could inhibit HIV gene expression (GFP fluorescence) in primary peripheral blood lymphocytes (PBLs), which are natural targets for HIV-1 infection. The frequency of GFP-expressing cells was markedly reduced in cells transfected with homologous siRNAs (T98, G388, nef) relative to cells transfected with mismatched siRNAs or non-transfected cells (FIG. 1E). The level of HIV_{NL-GFP} RNA, as determined by polymerase chain reaction with reverse transcription (RT-PCR), was also markedly reduced in cells transfected with homologous siRNAs (results not shown). Therefore, the components of siRNA-activated RNAi are fully functional in cells naturally targeted by HIV-1 infection.

### Example IV

### siRNA Degradation of Genomic Viral HIV RNA Associated with Viral Proteins

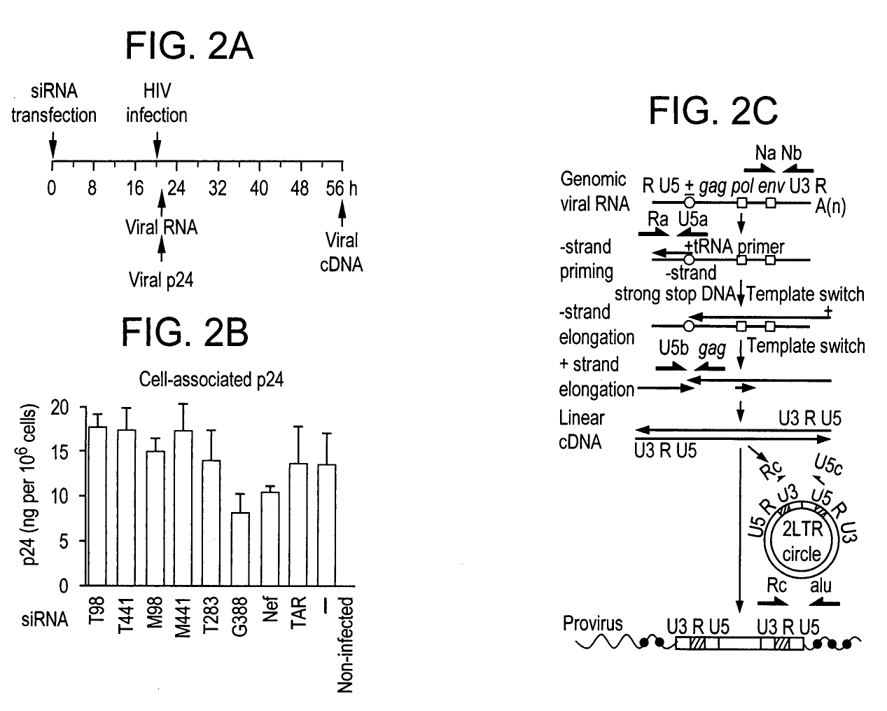

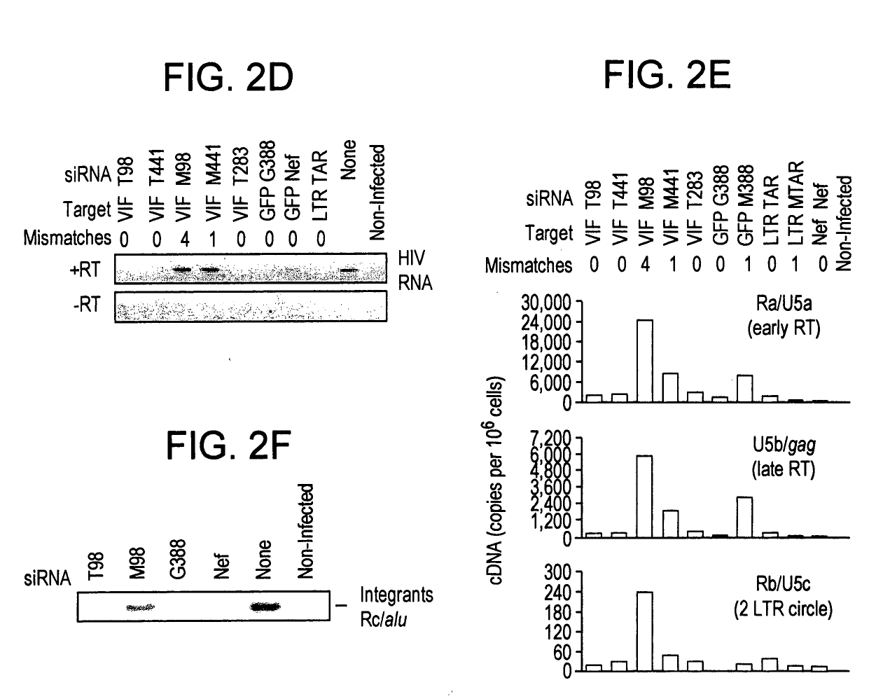

Upon HIV-1 infection, genomic viral RNA is introduced into the host cell cytoplasm in the form of a nucleoprotein complex, which comprises viral proteins in association with genomic viral RNA (Moore, J. & Stevenson, M., Nature Rev. Mol. Cell Biol. 1, 40-49 (2000). Within this complex, the viral reverse transcriptase enzyme directs the synthesis of viral cDNA intermediates from the genomic viral RNA template. Recent studies with RSV have indicated that genomic viral RNA, which is tightly associated with nucleocapsid protein, is resistant to siRNAs (Bitko, V. & Barik, S., J. Cell Biochem. 80, 441-454 (2000)). Whether siRNAs were able to direct the specific degradation of genomic viral RNA of HIV-1 was investigated. The experimental design is outlined in FIG. 2A. Magi cells were transfected with the various siRNAs and infected with HIV_{NL-GFP} 20 hours later. Transfection of cells with siRNAs did not significantly interfere with virus uptake per se, on the basis of levels of cell-associated p24 at 1 hour after infection (FIG. 2B). The strategy for analysis of viral reverse-transcription intermediates in acutely infected cells is outlined in FIG. 2C. At 1 hour after infection, genomic viral RNA was specifically detected in cells transfected with mismatched siRNAs and in non-transfected cells (M98, M441), but not in cells transfected with homologous siRNAs (FIG. 2D). Because genomic viral RNA is the template for the synthesis of viral cDNA intermediates, the synthesis of viral cDNAs, determined 36 hours after infection, was dramatically inhibited in cells transfected with homologous siRNAs (T98, GFP, nef) (FIG. 2E). Small interfering RNAs bearing one-nucleotide mismatch (M441, M388) were partially inhibitory relative to the siRNA bearing four mismatches (FIG. 2E). Small interfering RNAs were quite stable in cells: HIV entry was suppressed to equal levels whether virus was added 20 hours or 4 days after siRNA transfection (data not shown).

### Example V

### siRNAs Interrupt Early Events in the HIV Replication Cycle, Preventing Synthesis of Viral Reverse-transcription Intermediates and Establishment of Provirus

Upon completion of viral cDNA synthesis, viral sequences integrate into cellular DNA to form a provirus. The level of provirus formation, as evidenced by the presence of junction sequences flanking viral and cellular DNA (FIG. 2E), was markedly reduced in cells transfected with homologous siRNAs (T98, G388, nef) relative to cells transfected with mismatched (M98) siRNAs or non-transfected cells (FIG. 2F). Collectively, these studies indicate that siRNAs interrupt early events in the HIV replication cycle by directing the specific degradation of genomic HIV-1 RNA, thereby preventing the subsequent synthesis of viral reverse-transcription intermediates and establishment of the provirus.

### Example VI

### Inhibition of HIV with Expressed siRNAs

Expression of siRNAs from plasmid templates offers several advantages over synthetic siRNAs, such as stable selection under selectable markers and inducible promoters, which are features that could be useful for genetic approaches to HIV therapy. Thus, whether expressed siRNAs could inhibit HIV was examined. Modifying a strategy used previously in plants (Wang, M. B. & Waterhouse, P. M., Plant. Mol. Biol. 43, 67-82 (2000); Varshawesley, S., et al., Plant J. 27, 581-590 (2001)), plasmids were constructed containing a 19-base pair (bp) region of the HIV-1 vif gene in 5′-3′ and 3′-5′ orientations under the control of a T7 promoter (FIG. 3A). Virus production was determined 24 hours after a three-way transfection of Magi cells with an HIV_{NL-GFP} molecular clone, the linearized vif hairpin plasmid (T1 Vif) and a vector expressing T7 RNA polymerase (T7 pol). In the presence of T7 RNA polymerase, T7 transcripts derived from BstBI-linearized expression plasmids would be predicted to comprise a GGUACC sequence from the T7 promoter, a 19-bp stem of self-complementary vif sequences, a 3-, 5- or 7-nucleotide loop and a 3′ UU overhang. All three vif hairpin plasmids containing 3-, 5- or 7-nucleotide loops potently suppressed virus production to 20-30-fold relative to non-transfected cells. By comparison, the presence of an identical plasmid lacking vif sequences (TL Δ vif or a control plasmid pcDNA) had no effect on virus production in co-transfected cells (FIG. 3B). This inhibitory effect on virus production was reflected by a loss of viral RNA (FIG. 3C).

### Example VII

### Inhibition of HIV with Expressed siRNAs in Primary Lymphocytes

The vif hairpin plasmid (TL vif7) also inhibited viral gene expression in primary lymphocytes, whereas there was no inhibitory effect of the plasmid lacking vif sequences in these cells (FIG. 3D). These results indicate that a sequence-specific RNAi effect can be activated in established and primary cells by siRNAs derived from self-complementary hairpin-generating plasmids. This provides a rationale for gene-therapy approaches to HIV that complement existing post-transcriptional approaches for inhibiting HIV, including ribozymes and antisense RNA (Domburg, R. & Pomerantz, R. J., Adv. Pharmacol. 49, 229-261 (2000)).

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A small interfering RNA (siRNA) comprising a sequence sufficiently complementary to a portion of the HIV genome to mediate RNA interference (RNAi).

2. The siRNA of claim 1, wherein the siRNA is between about 15 and about 25 nucleotides long.

3. The siRNA of claim 1, wherein the siRNA is between about 20 and about 23 nucleotides long.

4. The siRNA of claim 1, wherein the siRNA comprises a sequence sufficiently complementary to a Long Terminal Repeats (LTR) region of the HIV genome to mediate RNAi.

5. The siRNA of claim 1, wherein the siRNA comprises a sequence sufficiently complementary to a nef gene of the HIV genome to mediate RNAi.

6. The siRNA of claim 1, wherein the siRNA comprises a sequence sufficiently complementary to a vif gene of the HIV genome to mediate RNAi.

7. The siRNA of claim 1, wherein the siRNA comprises a sequence sufficiently complementary to a gene of the HIV genome that codes for a reverse transcriptase enzyme to mediate RNAi.

8. The siRNA of claim 1, wherein the siRNA comprises a sequence sufficiently complementary to a gene of the HIV genome that codes for a capsid protein or an envelope protein to mediate RNAi.

9. The siRNA of claim 1, wherein the siRNA is an expressed siRNA.

10. The siRNA of claim 1, wherein the siRNA is a synthetic siRNA.

11. The siRNA of claim 10, wherein the siRNA is a synthetic 21-nucleotide siRNA.

12. The siRNA of claim 1, wherein the siRNA is a short hairpin siRNA (shRNA).

13. The siRNA of claim 1, wherein the siRNA is a short hairpin siRNA (shRNA) expressed from a plasmid.

14. The siRNA of claim 1, wherein the siRNA inhibits synthesis of viral HIV cDNA.

15. The siRNA of claim 1, wherein the siRNA promotes the degradation of or inhibits synthesis of viral HIV cDNA intermediates.

16. The siRNA of claim 1, wherein the siRNA promotes the degradation of or inhibits synthesis of genomic viral HIV RNA.

17. The siRNA of claim 1, wherein the siRNA mediates RNAi during an early viral replication cycle event.

18. The siRNA of claim 1, wherein the siRNA mediates RNAi during a late viral replication cycle event.

19. The siRNA of claim 1, wherein the siRNA is generated by endonuclease cleavage of dsRNA.

20. The siRNA of claim 1, wherein the siRNA is modified by the substitution of at least one nucleotide with a modified nucleotide.

21. The siRNA of claim 1, wherein the siRNA has at least one mismatch when compared to the sequence of the HIV genome.

22. A siRNA complex comprising:
the siRNA of claim 1; and
one or more proteins associated with the siRNA that recognize the portion of the HIV genome.

23. A method of treating a subject infected with HIV, the method comprising the steps of:
providing an siRNA comprising a sequence sufficiently complementary to a portion of the HIV genome to mediate RNA interference (RNAi); and
initiating RNAi by administering the siRNA to said subject.

24. The method of claim 23, comprising the step of providing a siRNA complex comprising:
the siRNA comprising a sequence sufficiently complementary to a portion of the HIV genome to mediate RNA interference (RNAi); and
one or more proteins associated with the siRNA that recognize the portion of the HIV genome.

25. The method of claim 23 comprising the step of providing a siRNA complex comprising the siRNA.

26. The method of claim 23 comprising the steps of:
analyzing a portion of an HIV genome present in the subject; and
providing an siRNA comprising a sequence sufficiently complementary to the portion of the HIV genome present in the subject to mediate RNAi.

27. The method of claim 23 comprising the steps of:
analyzing a portion of an HIV genome, for each of a plurality of mutated HIV genomes present in the subject; and
providing one or more siRNAs comprising a sequence sufficiently complementary to the portion of the HIV genome, for each of the plurality of mutated HIV genomes present in the subject.

28. A method of inhibiting or preventing HIV replication or infection in a subject, the method comprising the steps of:
providing a siRNA comprising a sequence sufficiently complementary to a portion of the HIV genome to mediate RNA interference (RNAi); and
administering the siRNA to the subject the siRNA such that HIV replication or infection is inhibited or prevented.

29. The method of claim 28 wherein the siRNA is expressed from a vector template.

30. The method of claim 28, wherein viral RNA is degraded in the early stages of replication such that provirus formation is inhibited or prevented.

31. The method of claim 28, wherein viral RNA is degraded in the late stages of replication such that release of newly formed viral RNA is inhibited or prevented.

32. The method of claim 28 comprising the steps of:
analyzing a portion of an HIV genome present in the subject; and
providing an siRNA comprising a sequence sufficiently complementary to the portion of the HIV genome present in the subject to mediate RNAi.

33. The method of claim 28 comprising the steps of:
analyzing a portion of an HIV genome, for each of a plurality of mutated HIV genomes present in the subject; and
providing one or more siRNAs comprising a sequence sufficiently complementary to the portion of the HIV genome, for each of the plurality of mutated HIV genomes present in the subject.

34. A method of inhibiting or preventing HIV replication or infection in a cell, the method comprising the steps of:
providing a siRNA comprising a sequence sufficiently complementary to a portion of the HIV genome to mediate RNA interference (RNAi); and
inhibiting or preventing HIV replication or infection by contacting a cell with the siRNA.

35. The method of claim 34, wherein the siRNA is expressed from a vector.

36. The method of claim 34, wherein viral RNA is degraded in the early stages of replication such that provirus formation is inhibited or prevented.

37. The method of claim 34, wherein viral RNA is degraded in the late stages of replication such that release of newly formed viral RNA from the cell is inhibited or prevented.

38. The method of claim 34, comprising the step of providing a cell unexposed to the HIV virus.

39. The method of claim 34, comprising the step of providing a cell comprising less than 500 copies of viral HIV RNA.

40. The method of claim 34, comprising the step of providing a cell comprising less than 1000 copies of viral HIV RNA prior to contacting the cell with the siRNA.

41. The method of claim 34, comprising the step of providing a cell exposed to HIV, but wherein the HIV RNA has not integrated into the cell genome.

42. The method of claim 34, wherein said cell is a lymphocyte.

43. The method of claim 42, wherein said lymphocyte is a primary peripheral blood lymphocyte.

44. The method of claim 34, wherein the siRNA is expressed from a vector template in vivo.

45. A vector that expresses an siRNA comprising a sequence sufficiently complementary to a portion of the HIV genome to mediate RNA interference (RNAi).

46. The vector of claim 45, wherein the siRNA is a shRNA.

47. The vector of claim 45 wherein the vector expresses a plurality of siRNAs comprising sequences sufficiently complementary to portions of the HIV genome to mediate RNAi.

48. The vector of claim 47 wherein at least one of the siRNAs is a shRNA.

49. The vector of claim 47 wherein the plurality of siRNAs comprise sequences sufficiently complementary to staggered portions of the HIV genome to mediate RNAi.

50. The vector of claim 47 wherein the plurality of siRNAs comprise sequences sufficiently complementary to different genes in the HIV genome.

51. The vector of claim 47 wherein the plurality of siRNAs comprise at least three sequences sufficiently complementary to one or more regions of the HIV genome selected from the group consisting of: a region coding for reverse transcriptase, a region coding for protease, and a vif gene.

52. The vector of claim 47 wherein the plurality of siRNAs comprise at least five sequences sufficiently complementary to one or more regions of the HIV genome selected from the group consisting of: a region coding for reverse transcriptase, a region coding for protease, a tat gene, a rev gene, and a vif gene.

53. The vector of claim 47 wherein the plurality of siRNAs comprise sequences sufficiently complementary to one or more regions of the HIV genome selected from the group consisting of: a region coding for reverse transcriptase, a region coding for protease, a tat gene, a rev gene, and a vif gene, a gag gene, a vpr gene, a region coding for an envelope protein, a region coding for a capsid protein, and a LTR region.

54. The vector of claim 47 wherein the vector is a plasmid vector.

55. The vector of claim 47 wherein the vector is a viral vector.

56. A method of treating a subject infected with HIV, the method comprising the steps of:
providing the vector of claim 45; and
initiating RNA interference by administering the vector to said subject.

57. The method of claim 56, wherein viral RNA is degraded in the early stages of replication such that provirus formation is inhibited or prevented.

58. The method of claim 56, wherein viral RNA is degraded in the late stages of replication such that release of newly formed viral RNA is inhibited or prevented.

59. The method of claim 56 comprising the steps of:
analyzing a portion of an HIV genome present in the subject; and
providing an siRNA comprising a sequence sufficiently complementary to the portion of the HIV genome present in the subject to mediate RNAi.

60. The method of claim 56 comprising the steps of:
analyzing a portion of an HIV genome, for each of a plurality of mutated HIV genomes present in the subject; and
providing one or more siRNAs comprising a sequence sufficiently complementary to the portion of the HIV genome, for each of the plurality of mutated HIV genomes present in the subject.

61. A method of inhibiting or preventing HIV replication or infection in a subject, the method comprising the steps of:
providing the vector of claim 45; and
initiating RNA interference by administering the vector to said subject.

62. The method of claim 61, wherein viral RNA is degraded in the early stages of replication such that provirus formation is inhibited or prevented.

63. The method of claim 61, wherein viral RNA is degraded in the late stages of replication such that release of newly formed viral RNA is inhibited or prevented.

64. The method of claim 61 comprising the steps of:
analyzing a portion of an HIV genome present in the subject; and
providing an siRNA comprising a sequence sufficiently complementary to the portion of the HIV genome present in the subject to mediate RNAi.

65. The method of claim 61 comprising the steps of:
analyzing a portion of an HIV genome, for each of a plurality of mutated HIV genomes present in the subject; and
providing one or more siRNAs comprising a sequence sufficiently complementary to the portion of the HIV genome, for each of the plurality of mutated HIV genomes present in the subject.

66. A method of inhibiting or preventing HIV replication or infection in a cell, the method comprising the steps of:
providing the vector of claim 45; and
initiating RNA interference by administering the vector to said cell.

67. The method of claim 66, wherein viral RNA is degraded in the early stages of replication such that provirus formation is inhibited or prevented.

68. The method of claim 66, wherein viral RNA is degraded in the late stages of replication such that release of newly formed viral RNA from the cell is inhibited or prevented.

69. The method of claim 66, comprising the step of providing a cell unexposed to the HIV virus.

70. The method of claim 66, comprising the step of providing a cell comprising less than 500 copies of viral HIV RNA.

71. The method of claim 66, comprising the step of providing a cell comprising less than 1000 copies of viral HIV RNA prior to contacting the cell with the siRNA.

72. The method of claim 66, comprising the step of providing a cell exposed to HIV, but wherein the HIV RNA has not integrated into the cell genome.

73. The method of claim 66, wherein said cell is a lymphocyte.

74. The method of claim 73, wherein said lymphocyte is a primary peripheral blood lymphocyte.


SEQUENCE LISTING (20 sequences)

SEQ ID NO: 1 - DNA, 21 bp, Artificial Sequence
  siRNA oligonucleotide
ggaaagcuaaggacugguutt

SEQ ID NO: 2 - DNA, 21 bp, Artificial Sequence
  siRNA oligonucleotide
agcacacaaguagacccugtt

SEQ ID NO: 3 - DNA, 21 bp, Artificial Sequence
  siRNA oligonucleotide
cuuggcacuagcagcauuatt

SEQ ID NO: 4 - DNA, 20 bp, Artificial Sequence
  siRNA oligonucleotide
gaaagcuaggggaugguutt

SEQ ID NO: 5 - DNA, 21 bp, Artificial Sequence
  RNA molecule with two deoxythymidines at 3′ end
cuuggcacuaacagcauuatt

SEQ ID NO: 6 - RNA, 19 nt, Artificial Sequence
  siRNA oligonucleotide
gacuucaaggaagauggca

SEQ ID NO: 7 - DNA, 21 bp, Artificial Sequence
  siRNA oligonucleotide
gacuucaagggagauggcatt

SEQ ID NO: 8 - DNA, 21 bp, Artificial Sequence
  siRNA oligonucleotide
gugccuggcuagaagcacatt

SEQ ID NO: 9 - DNA, 21 bp, Artificial Sequence
  siRNA oligonucleotide
agaccagaucugagccuggtt

SEQ ID NO: 10 - DNA, 21 bp, Artificial Sequence
  siRNA oligonucleotide
agaccagauaugagccuggtt

SEQ ID NO: 11 - DNA, 22 bp, Artificial Sequence
  primer
tagaccagatctgagcctggga

SEQ ID NO: 12 - DNA, 22 bp, Artificial Sequence
  primer
gtagttctgccaatcagggaag

SEQ ID NO: 13 - DNA, 21 bp, Artificial Sequence
  primer
gtctgagggatctctagttac

SEQ ID NO: 14 - DNA, 19 bp, Artificial Sequence
  primer
gggagctctctggctaact

SEQ ID NO: 15 - DNA, 20 bp, Artificial Sequence
  primer
ggattaactgcgaatcgttc

SEQ ID NO: 16 - DNA, 17 bp, Artificial Sequence
  primer
gacagggcttggaaagg

SEQ ID NO: 17 - DNA, 20 bp, Artificial Sequence
  primer
ttagcagttctgaagtactc

SEQ ID NO: 18 - DNA, 21 bp, Artificial Sequence
  primer
tgctgggattacaggcgtgag

SEQ ID NO: 19 - DNA, 21 bp, Artificial Sequence
  primer
ggctaactagggaacccactg

SEQ ID NO: 20 - DNA, 24 bp, Artificial Sequence
  primer
ctgctagagattttccacactgac